(19) United States
(12) Patent Application Publication
(10) Pub. No.: US 2021/0324382 A1
(43) Pub. Date: Oct. 21, 2021

(54) Chimeric DNA:RNA Guide for High Accuracy Cas9 Genome Editing

(71) Applicant: Massachusetts Institute of Technology, Cambridge, MA (US)

(72) Inventors: Noah Jakimo, Boston, MA (US); Pranam Chatterjee, Cambridge, MA (US); Joseph M. Jacobson, Newton, MA (US)

(73) Assignee: Massachusetts Institute of Technology, Cambridge, MA (US)

(21) Appl. No.: 17/156,492

(22) Filed: Jan. 22, 2021

Related U.S. Application Data
(63) Continuation of application No. 15/820,425, filed on Nov. 21, 2017, now abandoned.
(60) Provisional application No. 62/425,041, filed on Nov. 21, 2016.

(51) Int. Cl. C12N 15/11; C12N 9/22; C12N 15/90; C12N 9/96

(52) U.S. Cl. CPC C12N 15/11; C12N 9/22; C12N 15/907; C12N 9/96; C12N 15/1136; C12N 2310/335; C12N 2310/20; C12N 2310/343; C12N 15/111

(57) ABSTRACT

A chimeric DNA:RNA guide for very high accuracy Cas9 genome editing employs nucleotide-type substitutions in nucleic acid-guided endonucleases for enhanced specificity. The CRISPR-Cas9 gene editing system is manipulated to generate chimeric DNA:RNA guide strands to minimize the off-target cleavage events of the S. pyogenes Cas9 endonuclease. A DNA:RNA chimeric guide strand is sufficient to guide Cas9 to a specified target sequence for indel formation and minimize off-target cleavage events due to the specificity conferred by DNA-DNA interactions. Use of chimeric mismatch-evading lowered-thermostability guides (“melt-guides”) demonstrate that nucleotide-type substitutions in the spacer can reduce cleavage of sequences mismatched by as few as a single base pair. The chimeric mismatch-evading lowered-thermostability guides replace most gRNA spacer positions with DNA bases to suppress mismatched targets under Cas9's catalytic threshold.

### RELATED APPLICATIONS

This application is a continuation of U.S. patent application Ser. No. 15/820,425, filed Nov. 21, 2017, which claims the benefit of U.S. Provisional Application Ser. No. 62/425,041, filed Nov. 21, 2016, the entire disclosure of which is herein incorporated by reference.

### STATEMENT REGARDING FEDERALLY SPONSORED RESEARCH OR DEVELOPMENT

This invention was made with U.S. Government Support under Grant Number HR0011-16-2-0003, awarded by the Defense Advanced Projects Research Agency. The U.S. Government has certain rights in this invention.

### FIELD OF THE TECHNOLOGY

The present invention relates to genome editing and, in particular, to a DNA:RNA guide for a CRISPR-Cas9 genome editing system.

### BACKGROUND

An efficient, reliable mechanism of making precise, targeted changes to the genomes of living cells is a critical goal of biomedicine. The CRISPR-Cas9 genome editing system, where a short RNA strand (sgRNA) guides the Cas9 enzyme to a specific target sequence for double-stranded DNA cleavage [Jinek, et al. A programmable dual-RNA-guided DNA endonuclease in adaptive bacterial immunity. Science 337, 816-821 (2012)], has thus generated considerable excitement. However, the targeting and cleavage activity of Cas9 often results in off-target DNA modifications, which seriously limits its applications [Mali, et al., “CAS9 transcriptional activators for target specificity screening and paired nickases for cooperative genome engineering”, Nature Biotechnology 31, 833-838 (2013)]. There have been several studies aimed at improving DNA off-target cleavage, including a double-nicking approach consisting of using the nickase variant of Cas9 with a pair of offset sgRNAs properly positioned on the target DNA2, truncated guide RNAs utilized in conjunction with the double nicking strategy [Fu, et al., “Improving CRISPR-Cas nuclease specificity using truncated guide RNAs”, Nature Biotechnology 32, 279-284 (2014)], and an RNA-guided dCas9 fused to the FokI nuclease where two fused dCas9-FokI monomers can simultaneously bind target sites at a defined distance apart [Guilinger, et al., “Fusion of catalytically inactive Cas9 to FokI nuclease improves the specificity of genome modification”, Nature Biotechnology 32, 577-582 (2014); Tsai, et al., “Dimeric CRISPR RNA-guided FokI nucleases for highly specific genome editing”, Nature Biotechnology 32, 569-576 (2014)]. Few approaches, however, focus specifically on the strand invasion of the sgRNA and its stability within the target DNA.

Recent discoveries, characterizations, and modifications of natural nucleic acid-guided endonucleases (NGEns), such as CRISPR and RNAi, have resulted in their widespread use for genome editing, probing, and regulation [Wilson, R. C. & Doudna, J. A., “Molecular mechanisms of RNA interference”, Annual review of biophysics 42, 217-239 (2013); Sander, J. D. & Joung, J. K., “CRISPR-Cas systems for editing, regulating and targeting genomes”, Nature Biotechnology 32(4), 347-355 (2014); Mohr, S. E.; Smith, J. A.; Shamu, C. E.; Neumülller, R. A. & Perrimon, N., “RNAi screening comes of age: improved techniques and complementary approaches”, Nature reviews. Molecular cell biology 15, 591-600 (2014); Dominguez, A. A.; Lim, W. A. & Qi, L. S., “Beyond editing: repurposing CRISPR-Cas9 for precision genome regulation and interrogation”, Nature reviews. Molecular cell biology 17, 5-15 (2016)]. Unlike prior work on engineering modular proteins to recognize double-stranded DNA, NGEns are significantly less expensive to assemble and more straightforward to design, while maintaining similar specificity for the intended genomic target [Gaj, T.; Gersbach, C. A. & Barbas, C. F., “ZFN, TALEN, and CRISPR/Cas-based methods for genome engineering”, Trends in biotechnology 31, 397-405 (2013); Ul Ain, Q.; Chung, J. Y. & Kim, Y.-H., “Current and future delivery systems for engineered nucleases: ZFN, TALEN and RGEN”, Journal of controlled release: official journal of the Controlled Release Society 205, 120-127 (2015)].

Since applications like gene therapy may demand negligible off target effects, many efforts have focused on new mechanisms to control and tune the binding and cleavage of NGEns [Tsai, S. Q.; Zheng, Z.; Nguyen, N. T.; Liebers, M.; Topkar, V. V.; Thapar, V.; Wyvekens, N.; Khayter, C.; Iafrate, A. J.; Le, L. P.; Aryee, M. J. & Joung, J. K., “GUIDE-seq enables genome-wide profiling of off-target cleavage by CRISPR-Cas nucleases”, Nature biotechnology 33, 187-197 (2015); Hu, J. H.; Davis, K. M. & Liu, D. R., “Chemical Biology Approaches to Genome Editing: Understanding, Controlling, and Delivering Programmable Nucleases”, Cell chemical biology 23, 57-73 (2016)]. Several approaches include coupling the activity of the system to chemical or physical stimuli, requiring co-localized concurrent recognition of targets, or making subtle biochemical modifications that influence recognition kinetics [Ran, F. A.; Hsu, P. D.; Lin, C.-Y.; Gootenberg, J. S.; Konermann, S.; Trevino, A. E.; Scott, D. A.; Inoue, A.; Matoba, S.; Zhang, Y. & Zhang, F., “Double nicking by RNA-guided CRISPR Cas9 for enhanced genome editing specificity”, Cell 154, 1380-1389 (2013); Tsai, S. Q.; Zheng, Z.; Nguyen, N. T.; Liebers, M.; Topkar, V. V.; Thapar, V.; Wyvekens, N.; Khayter, C.; Iafrate, A. J.; Le, L. P.; Aryee, M. J. & Joung, J. K., “GUIDE-seq enables genome-wide profiling of off-target cleavage by CRISPR-Cas nucleases”, Nature biotechnology 33, 187-197 (2015); Zetsche, B.; Volz, S. E. & Zhang, F., “A split-Cas9 architecture for inducible genome editing and transcription modulation”, Nature biotechnology 33, 139-142 (2015); Jain, P. K.; Ramanan, V.; Schepers, A. G.; Dalvie, N. S.; Panda, A.; Fleming, H. E. & Bhatia, S. N., “Development of Light-Activated CRISPR Using Guide RNAs with Photocleavable Protectors”, Angewandte Chemie (International ed. in English) 55, 12440-12444 (2016)].

Several strategies for influencing recognition kinetics have exploited the sequential hybridization between an NGEn's invading guide and one strand of the DNA target (target strand), which is incrementally displaced from the non-target strand [Fu, Y.; Reyon, D. & Joung, J. K., “Targeted genome editing in human cells using CRISPR/Cas nucleases and truncated guide RNAs”, Methods in enzymology 546, 21-45 (2014); Liu, Y.; Zhan, Y.; Chen, Z.; He, A.; Li, J.; Wu, H.; Liu, L.; Zhuang, C.; Lin, J.; Guo, X.; Zhang, Q.; Huang, W. & Cai, Z., “Directing cellular information flow via CRISPR signal conductors”, Nature methods 13, 938-944 (2016)]. Such three-stranded structures are referred to as R-loops, when the invading strand is RNA, and D-loops, when the invading strand is DNA. The size of an R-loop and D-loop can serve as an allosteric switch for an NGEns cleavage activity [Kiani, S.; Chavez, A.; Tuttle, M.; Hall, R. N.; Chari, R.; Ter-Ovanesyan, D.; Qian, J.; Pruitt, B. W.; Beal, J.; Vora, S. et al., “Cas9 gRNA engineering for genome editing, activation and repression”, Nature methods 12(11), 1051-1054 (2015); Lim, Y.; Bak, S. Y.; Sung, K.; Jeong, E.; Lee, S. H.; Kim, J.-S.; Bae, S. & Kim, S. K., “Structural roles of guide RNAs in the nuclease activity of Cas9 endonuclease”, Nature Communications 7, 13350 (2016)]. Since an NGEn endonuclease like Cas9 partially stabilizes the R-loop, efforts have identified Cas9 variants with reduced R-loop stabilization and increased reliance on RNA-DNA base-pairing energies to maintain and extend the R-loop [Slaymaker, I. M.; Gao, L.; Zetsche, B.; Scott, D. A.; Yan, W. X. & Zhang, F., “Rationally engineered Cas9 nucleases with improved specificity”, Science 351, 84-88 (2016)]. Similarly, other efforts have either extended or truncated the NGEn's guide to modify the R-loop's stability [Josephs, E. A.; Kocak, D. D.; Fitzgibbon, C. J.; McMenemy, J.; Gersbach, C. A. & Marszalek, P. E., “Structure and specificity of the RNA-guided endonuclease Cas9 during DNA interrogation, target binding and cleavage”, Nucleic acids research 43, 8924-8941 (2015)].

Oligonucleotide-guided nucleases (OGNs) have enabled rapid advances in precision genome engineering. Though much effort has gone into characterizing and mitigating mismatch tolerance for the most widely adopted OGN, Streptococcus pyogenes Cas9 (SpCas9), potential off-target interactions may still limit applications where on-target specificity is critical. The recent discoveries, characterizations, and modifications of natural oligonucleotide-guided nucleases associated with CRISPR and RNAi have empowered a genome-editing revolution [Jinek, M. et al., “RNA-programmed genome editing in human cells”, eLife 2, e00471 (2013); Mali, P. et al., “RNA-guided human genome engineering via Cas9”, Science 339, 823-826 (2013); Cong, L. et al., “Multiplex genome engineering using crispr/cas systems”, Science 339, 819-823 (2013); Komor, A. C., Badran, A. H. & Liu, D. R., “CRISPR-based technologies for the manipulation of eukaryotic genomes”, Cell 168, 20-36 (2017)]. Low barriers for OGNs' cost and design drive their widespread adoption over alternatives, including modular base-recognition domains (i.e., transcription activator like effector, zinc finger, and pumilio assemblies), which can be hard to synthesize, or meganucleases, which are difficult to engineer for new targets [Reyon, D. et al., “Flash assembly of talens for high-throughput genome editing”, Nature biotechnology 30, 460-465 (2012); Ramirez, C. L. et al., “Unexpected failure rates for modular assembly of engineered zinc fingers”, Nature methods 5, 374-375 (2008); Adamala, K. P., Martin-Alarcon, D. A. & Boyden, E. S., “Programmable RNA-binding protein composed of repeats of a single modular unit”, Proceedings of the National Academy of Sciences 113, E2579-E2588 (2016); Takeuchi, R., Choi, M. & Stoddard, B. L., “Redesign of extensive protein—DNA interfaces of meganucleases using iterative cycles of in vitro compartmentalization”, Proceedings of the National Academy of Sciences 111, 4061-4066 (2014)]. Unlike protein-directed systems, OGNs also permit employing predictable nucleic acid chemistry and biophysics to alternative features [Hendel, A. et al., “Chemically modified guide RNAs enhance CRISPR-Cas genome editing in human primary cells”, Nature biotechnology 33, 985-989 (2015); Jain, P. K. et al., “Development of light-activated CRISPR using guide RNAs with photocleavable protectors”, Angewandte Chemie (International ed. in English) 55, 12440-12444 (2016); Lee, K. et al., “Synthetically modified guide RNA and donor DNA are a versatile platform for CRISPR-Cas9 engineering”, eLife 6 (2017); Ui-Tei, K. et al., “Functional dissection of sirna sequence by systematic DNA substitution: modified sirna with a DNA seed arm is a powerful tool for mammalian gene silencing with significantly reduced off-target effect”, Nucleic acids research 36, 2136-2151 (2008)].

Among the most important properties dictating the usage of a nucleic acid recognition system is its specificity. Thus, the desire to identify new methods diminishing potentially toxic or detrimental off-target activity has prompted many to measure and improve mismatch discrimination for RNA-guided SpCas9—the most prevalent OGN [Schaefer, K. A. et al., “Unexpected mutations after CRISPR-Cas9 editing in vivo”, Nature methods 14, 547-548 (2017); Tsai, S. Q. et al., “Circle-seq: a highly sensitive in vitro screen for genome-wide CRISPR-Cas9 nuclease off-targets”, Nature methods 14, 607-614 (2017); Doench, J. G. et al., “Optimized sgRNA design to maximize activity and minimize off-target effects of CRISPR-Cas9”, Nature biotechnology 34, 184-191 (2016)]. Up to now, others have increased its precision through broad approaches, such as controlling duration of exposure, enforcing co-localization on adjacent targets, or destabilizing binding affinity by minor variation [Davis, K. M., Pattanayak, V., Thompson, D. B., Zuris, J. A. & Liu, D. R., “Small molecule-triggered Cas9 protein with improved genome-editing specificity”, Nature chemical biology 11, 316-318 (2015); Ran, F. A. et al., “Double nicking by RNA-guided CRISPR Cas9 for enhanced genome editing specificity”, Cell 154, 1380-1389 (2013); Kleinstiver, B. P. et al., “High-fidelity CRISPR-Cas9 nucleases with no detectable genome-wide off-target effects”, Nature 529, 490-495 (2016); Fu, Y., Reyon, D. & Joung, J. K., “Targeted genome editing in human cells using CRISPR/Cas nucleases and truncated guide RNAs”, Methods in enzymology 546, 21-45 (2014)].

### SUMMARY

A chimeric DNA:RNA guide for very high accuracy Cas9 genome editing according to the invention employs nucleotide-type substitutions in nucleic acid-guided endonucleases for enhanced specificity. A goal is to develop novel DNA editing technologies to broaden the scope of genome engineering and to confer greater stability, minimize off-target DNA cleavage, and eliminate sequence restrictions for precision genetic manipulations within cells. A specific objective is to manipulate the current CRISPR-Cas9 gene editing system and generate chimeric DNA:RNA guide strands to minimize the off-target cleavage events of the S. pyogenes Cas9 endonuclease.

The work demonstrates that a DNA:RNA chimeric guide strand is sufficient to guide Cas9 to a specified target sequence for indel formation and minimize off-target cleavage events due to the specificity conferred by DNA-DNA interactions. The invention provides a new axis to control mismatch sensitivity along the recognition-conferring spacer sequence of SpCas9's guide RNA (gRNA). It introduces mismatch-evading lowered-thermostability guides (“melt-guides”) and shows how nucleotide-type substitutions in the spacer can reduce cleavage of sequences mismatched by as few as a single base pair. Upon co-transfecting melt-guides into human cell culture with an exonuclease involved in DNA repair, indel formation is observed on a standard genomic target at approximately 70% the rate of canonical gRNA and undetectable on off-target data.

The chimeric mismatch-evading lowered-thermostability guides replace most gRNA spacer positions with DNA bases to suppress mismatched targets under Cas9's catalytic threshold. As confirmed by in vitro cleavage assays, melt-guides can direct Cas9 with substantially enhanced mismatch discrimination. It has been verified in vivo that melt-guides according to the invention can achieve efficient mutagenesis with greater precision by providing deep sequencing data from transfected HEK293T cells stably expressing Cas9.

### BRIEF DESCRIPTION OF THE DRAWINGS

Other aspects, advantages and novel features of the invention will become more apparent from the following detailed description of the invention when considered in conjunction with the accompanying drawings, wherein:

FIG. 1 depicts nucleotide substitutions for spacer-DNA enhancement according to an example of one embodiment of the invention (SEQ ID NO. 8).

FIGS. 2A-C depict example results obtained from in vitro cleavage assays of on-target (FIG. 2A—SEQ ID NO. 1) and several off-target substrates (FIG. 2 A—SEQ ID NOs. 2-7) by ribonucleoprotein assemblies of a corresponding spacer-DNA enhancement melt-guide (FIG. 2B—SEQ ID NO. 9) complexed with tracrRNA (FIG. 2C—SEQ ID NO. 10) and purified S. pyogenes Cas9, according to one aspect of the invention.

FIGS. 3A-D depict an example cleavage assay gel for an embodiment obtained by reducing the RNA content of spacer-DNA enhancement melt-guide (FIG. 3B-SEQ ID NO. 11; FIG. 3C—SEQ ID NO. 12; FIG. 3D—SEQ ID NO. 13) to a single base in the spacer sequence, according to one aspect of the invention.

FIG. 4 depicts an example workflow to knockout a gene in cell culture stably expressing Cas9 and tracrRNA, according to one aspect of the invention (SEQ ID NOs. 14-16).

FIG. 5 depicts an example workflow for determining off-targets effects of guide molecules by introducing modifications to the Guide-seq protocol, according to one aspect of the invention.

FIG. 6 depicts an example annotated 3D structure of a target-guide-Cas9 R-loop based on PDB 5F9R shown above the 2D structure of a melt-guide, according to one aspect of the invention.

FIGS. 7A-B depict a model of relative R-loop expansion rate differences that increase mismatch sensitivity for melt-guides compared to gRNA (FIG. 7B—SEQ ID NO. 9).

FIGS. 8 and 9 depict Rosetta energy scores with DNA substitutions in bound and unbound structures from PDBs 4UN3 and 4ZTO.

FIG. 10 is an inverted contrast-adjusted gel image of 4-hour Cas9 in vitro digests of targets with mismatches ranging from 0 to 3 using gRNA or melt-guide.

FIGS. 11A-B depict gel images from Cas9 digests with melt-guides of on (SEQ ID NO. 17) and off-target (SEQ ID NO. 18) sequences for MX.

FIGS. 12A-B depict gel images from Cas9 digests with melt-guides of on (SEQ ID NO. 19) and off-target (SEQ ID NOs. 20, 21) sequences for FANCF.

FIGS. 13A-B depict gel images from Cas9 and eCas9 digests with melt-guides of on (SEQ ID NO. 22) and off-target (SEQ ID NOs. 23-26) sequences for VEGFA site 2.

FIG. 14 depicts gel images from Cas9 digests with melt-guides targeting VEGFA site 2 and Cas9 digests with additional UNA substitutions (SEQ ID NO. 27).

FIGS. 15A-B depict gel images from Cas9 digests with melt-guides targeting FANCF (SEQ ID NOs. 18-20) with 10 phosphorothioate (PS) bonds replacing phosodiester bonds (FIG. 15B—SEQ ID NO. 28).

FIGS. 16A-B depict gel images from Cas9 digests with melt-guides targeting VEGFA site 2 (SEQ ID NOs. 22-24) with 4 phosphorothioate (PS) bonds replacing phosodiester bonds (FIG. 16B—SEQ ID NO. 27).

FIGS. 17A-B are graphs showing Melt-guide and gRNA cleavage time courses of no-mismatch target with Cas9 plot alongside least-squares-fitting logarithmic functions.

FIGS. 18A-B are inverted contrast-adjusted gel images of short and long Cas9 digests of double-stranded and single-stranded targets with no mismatches (FIG. 18A) or none (FIG. 18B) using gRNA or melt-guides with spacers containing either all-DNA, 3 DNA distributed, or an additional 3 DNA fill-in.

FIG. 19 depicts a T7EI endonuclease assay on genomic VEGFA site 1 amplicons upon Cas9 mutagenesis with various melt-guide designs.

FIG. 20 is a dual y-axis chart showing deep sequencing indel measurements on-target and at a known off-target, comparing mutagenesis by gRNA to melt-guides designed with DNA substitutions in their first 11 positions with and without Trex2 overexpression.

FIG. 21 is an inverted gel image of Cas9 digest of no-mismatch VEGFA site 1 target with almost all-DNA melt-guide of crRNA-length (SEQ ID NO. 29).

FIGS. 22A-B depict a T7EI endonuclease assay on genomic EMX amplicons (FIG. 22A—SEQ ID NOs. 17, 18) and a reported off-target upon mutagenesis by lipofection of protein Cas9 pre-assembled with either gRNA or melt-guide (FIG. 22B—SEQ ID NO. 30).

### DETAILED DESCRIPTION

In one aspect of the invention, a chimeric DNA:RNA guide strand is employed to allow for sufficient strand invasion by the RNA motif, and then the less thermodynamically stable DNA-DNA interaction is subsequently utilized for increased specificity. The central premise is that the RNA component of the guide molecule is sufficient for strand invasion into the duplex DNA target, and the DNA motif will confer added specificity. This hypothesis was formulated due to relatively weaker DNA-DNA interactions, as compared to RNA-DNA interactions [Lesnik, et al., “Relative Thermodynamic Stability of DNA, RNA, and DNA:RNA Hybrid Duplexes: Relationship with Base Composition and Structure”, Biochemistry 34, 10807-10815 (1995)], which will require increased, if not complete, complementarity for hybridization and stabilization to the target DNA. To test the hypothesis, a design strategy was employed wherein the spacer sequence of the guide molecule primarily consists of DNA, while the spacer bases that interact with Cas9, along with the tracRNA sequence, remain as RNA, to maintain sufficient guide activity.

Human cell-based assays and Next Generation Sequencing (NGS) were utilized to assess successful targeting and quantify the frequency of insertion-deletion mutations and analyze off-target events. The rationale is that, by generating a more specific CRISPR-Cas9 gene editing system, off-target effects will be minimized to allow for safer usage in biomedical applications.

The central hypothesis was tested via the following specific aims: (1) Human cell-based assays to assess targeting and quantify frequency of insertion-deletion mutations. The working hypothesis was that a chimeric DNA:RNA guide will demonstrate comparable levels of insertion-deletion mutations and successful DNA targeting to previously developed CRISPR-Cas9 gene editing systems. To test the hypothesis, an EGFP disruption assay as well as T7 endonuclease I (T7EI) and Sanger sequencing assays were employed to assess successful targeting and quantify the frequency of insertion-deletion mutations. (2) Next Generation Sequencing for enabling high-throughput CRISPR validation and quantifying Off-Target cleavage events. The working hypothesis was that, due to added specificity conferred by DNA-DNA interactions at the spacer sequence on the target DNA, off-target events will be minimal compared to similar published CRISPR-Cas9 modified systems. To test this hypothesis, Next Generation Sequencing (NGS) was employed, which provides highly sensitive data on the exact nature of the genomic modifications being made.

In particular, the invention provides an alternative approach to lowering R-loop Tm for improving NGEn specificity, which is accomplished by substituting positions in nucleic acid guides with other nucleotide types or analogs.

In a preferred embodiment, the invention is a modification to nucleic acid-guided endonucleases (NGEns) such that native guide molecules types (e.g. ribonucleic acid (RNA) or deoxyribonucleic acid (DNA)) contain nucleotide-type substitutions for the purpose of lowering melting temperature with the target. For example, changing parts of an RNA guide's backbone and bases to DNA or changing parts of an RNA or DNA guide to unlocked nucleobase analogs (UNA) [Lesnik, E. A. & Freier, S. M., “Relative thermodynamic stability of DNA, RNA, and DNA:RNA hybrid duplexes: relationship with base composition and structure”, Biochemistry 34, 10807-10815 (1995)]. Within the context of toehold-based strand displacement probes, which have analogous kinetics to R/D-loop expansion, DNA/DNA duplexes were shown to have enhanced mismatch discrimination over RNA/DNA duplexes [Zhang, D. Y.; Chen, S. X. & Yin, P., “Optimizing the specificity of nucleic acid hybridization”, Nature Chemistry 4(3), 208-214 (2012)]. Specificity improvements were also demonstrated for UNA-RNA/RNA duplexes over RNA/RNA duplexes in work applied to RNA silencing [Vaish, N.; Chen, F.; Seth, S.; Fosnaugh, K.; Liu, Y.; Adami, R.; Brown, T.; Chen, Y.; Harvie, P.; Johns, R.; Severson, G.; Granger, B.; Charmley, P.; Houston, M.; Templin, M. V. & Polisky, B., “Improved specificity of gene silencing by siRNAs containing unlocked nucleobase analogs”, Nucleic acids research 39, 1823-1832 (2011)].

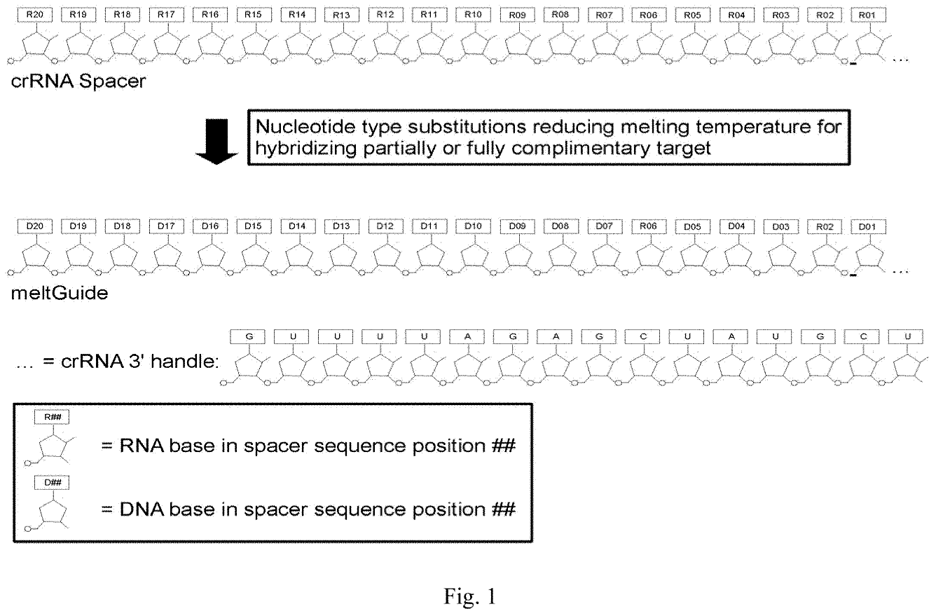

In a preferred embodiment, Cas9 and trans-activating CRISPR RNA (tracrRNA) from S. pyogenes form a complex with modified CRISPR RNA (crRNA) guide that has 17 of 20 bases in the crRNA's target-defining spacer region swapped with DNA nucleotides. Inferred by crystal structure of Cas9's contacts with crRNA's backbone, the second, fifth and sixth spacer bases away from the 3′ universal handle of crRNA for binding tracrRNA all remain as RNA [Nishimasu, H.; Ran, F. A.; Hsu, P. D.; Konermann, S.; Shehata, S. I.; Dohmae, N.; Ishitani, R.; Zhang, F. & Nureki, O., “Crystal structure of Cas9 in complex with guide RNA and target DNA”, Cell 156, 935-949 (2014)]. This embodiment is referred to as spacer-DNA enhancement (SpaDE). The described substitutions are illustrated in FIG. 1, which depicts nucleotide substitutions for spacer-DNA enhancement according to an example of one embodiment of the invention. The nucleotide type substitutions reduce the melting temperature for hybridizing a partially or fully complimentary target. More generally, embodiments of the invention may be referred to as guide molecules “modified to effect lowered Tm” (“melt-guides”).

Oligonucleotides with combinations of mixed nucleic acid types can be synthesized through phosphoramidite chemistry or enzymatic assembly and are commercially supplied by several vendors, including Integrated DNA Technologies (IDT), TriLink Biotechnologies and ATDBio. These methods and services also allow further modifications, such as 2′O Methyl RNA and phosphorothioate backbone linkages, which confer resistance to endonucleases and exonucleases, respectively, for increased cellular and in vivo lifetime of melt-guides [Hendel, A.; Bak, R. O.; Clark, J. T.; Kennedy, A. B.; Ryan, D. E.; Roy, S.; Steinfeld, I.; Lunstad, B. D.; Kaiser, R. J.; Wilkens, A. B.; Bacchetta, R.; Tsalenko, A.; Dellinger, D.; Bruhn, L. & Porteus, M. H., “Chemically modified guide RNAs enhance CRISPR-Cas genome editing in human primary cells”, Nature biotechnology 33, 985-989 (2015)]. A melt-guide can be engineered from an NGEn system with or without trans-activating nucleic acids (e.g. Cas9 and Cpf1, respectively). These trans-activating nucleic acids can be synthesized by the same methods as either separate molecules or chemically linked to the melt-guide.

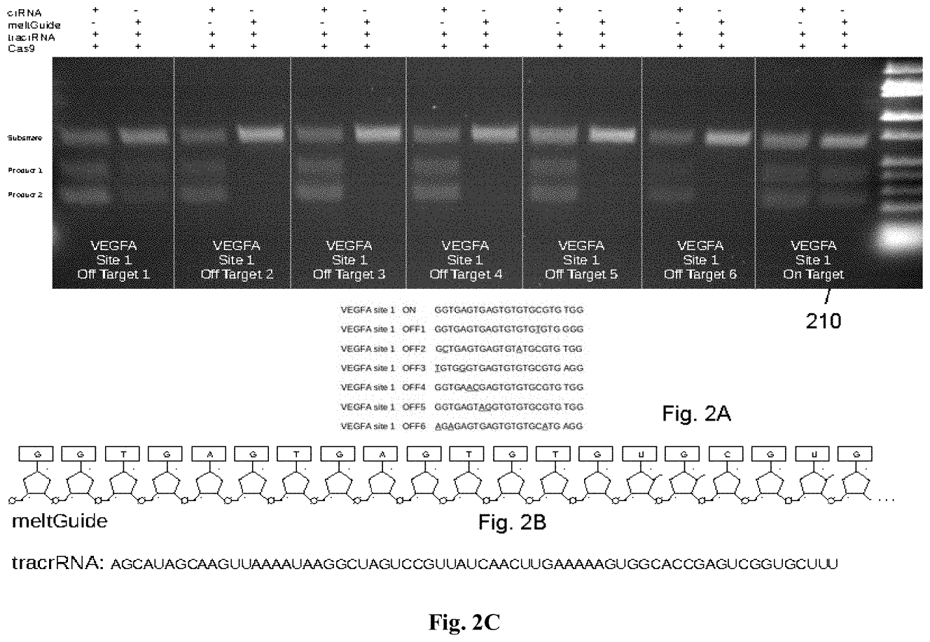

FIGS. 2A-C depict example results (FIG. 2A) obtained from in vitro cleavage assays of on-target 210 and several off-target substrates by ribonucleoprotein assemblies of a corresponding spacer-DNA enhancement melt-guide (FIG. 2B) complexed with tracrRNA (FIG. 2C) and purified S. pyogenes Cas9. In FIGS. 2A-C, reduction to practice is demonstrated by in vitro cleavage assays of on-target (from a sequence in the human VEGF-A gene commonly used as a specificity benchmark) and off-target substrates by ribonucleoprotein assemblies of a corresponding SpaDE melt-guide complexed with tracrRNA and purified S. pyogenes Cas9 (supplied by New England BioLabs). The electrophoresis gel image in FIG. 2A of DNA cleavage products and remaining substrate from 6 hours of digestion shows melt-guide directed cleavage on-target and virtually undetectable cleavage on five of six off-target substrates.

In addition to crystal structure, constraints on positions in an NGEn system's guide molecule that tolerate nucleotide type substitutions can also be derived empirically with a library of substitution variants [Nishimasu, H.; Cong, L.; Yan, W. X.; Ran, F. A.; Zetsche, B.; Li, Y.; Kurabayashi, A.; Ishitani, R.; Zhang, F. & Nureki, O., “Crystal Structure of Staphylococcus aureus Cas9”, Cell 162, 1113-1126 (2015); Hirano, H.; Gootenberg, J. S.; Horii, T.; Abudayyeh, O. O.; Kimura, M.; Hsu, P. D.; Nakane, T.; Ishitani, R.; Hatada, I.; Zhang, F.; Nishimasu, H. & Nureki, O., “Structure and Engineering of Francisella novicida Cas9”, Cell 164, 950-961 (2016); Yamano, T.; Nishimasu, H.; Zetsche, B.; Hirano, H.; Slaymaker, I. M.; Li, Y.; Fedorova, I.; Nakane, T.; Makarova, K. S.; Koonin, E. V.; Ishitani, R.; Zhang, F. & Nureki, O., “Crystal Structure of Cpf1 in Complex with Guide RNA and Target DNA”, Cell 165, 949-962 (2016); Miyoshi, T.; Ito, K.; Murakami, R. & Uchiumi, T., “Structural basis for the recognition of guide RNA and target DNA heteroduplex by Argonaute”, Nature Communications 7, 11846 (2016)].

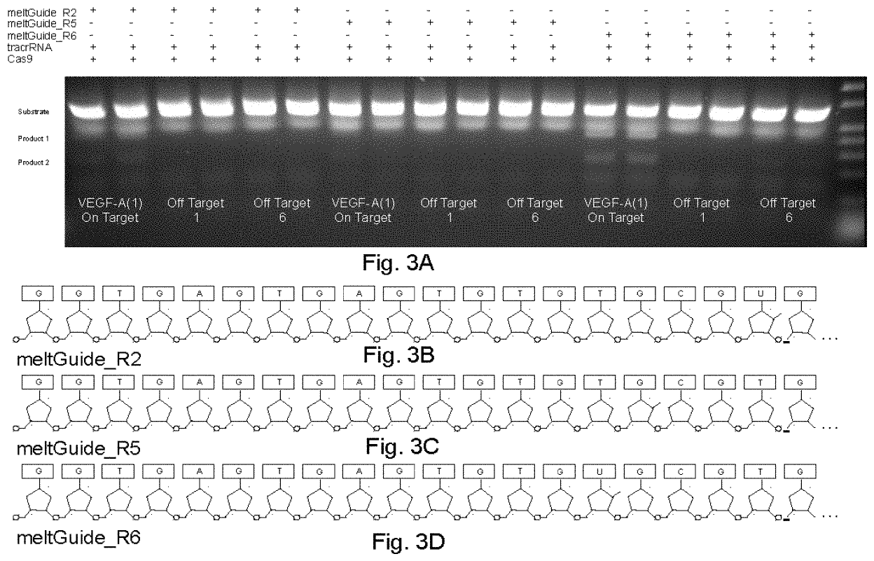

The cleavage assay gel shown in FIGS. 3A-D demonstrates such embodiments by depicting an embodiment obtained by reducing the RNA content of spacer-DNA enhancement (SpaDE) melt-guide to a single base in the spacer sequence. Such melt-guides show activity on-target and reduced relative activity on the off-target sequence that is cleaved when using the SpaDE melt-guide of FIG. 2B. Additional embodiments include melt-guide designs with secondary structure (e.g. hairpins), truncations, and base changes in the target-defining region for hybridization kinetics to further improve specificity. Embodiments can also include variants of the NGEn endonuclease for increased tolerance of melt-guide designs.

For in vitro applications, melt-guides are compatible with delivery methods used for NGEn systems, including but not limited to, electroporation, lipofection, cell-penetration, membrane perturbation, vesicle production, viral infection, and nanoparticle injection [Liang, X.; Potter, J.; Kumar, S.; Zou, Y.; Quintanilla, R.; Sridharan, M.; Carte, J.; Chen, W.; Roark, N.; Ranganathan, S., et al., “Rapid and highly efficient mammalian cell engineering via Cas9 protein transfection”, Journal of biotechnology 208, 44-53 (2015); Han, X.; Liu, Z.; Jo, M. C.; Zhang, K.; Li, Y.; Zeng, Z.; Li, N.; Zu, Y. & Qin, L., “CRISPR-Cas9 delivery to hard-to-transfect cells via membrane deformation”, Science advances 1, e1500454 (2015); Zuris, J. A.; Thompson, D. B.; Shu, Y.; Guilinger, J. P.; Bessen, J. L.; Hu, J. H.; Maeder, M. L.; Joung, J. K.; Chen, Z.-Y. & Liu, D. R., “Cationic lipid-mediated delivery of proteins enables efficient protein-based genome editing in vitro and in vivo”, Nature biotechnology 33, 73-80 (2015); Yin, H.; Song, C.-Q.; Dorkin, J. R.; Zhu, L. J.; Li, Y.; Wu, Q.; Park, A.; Yang, J.; Suresh, S.; Bizhanova, A.; Gupta, A.; Bolukbasi, M. F.; Walsh, S.; Bogorad, R. L.; Gao, G.; Weng, Z.; Dong, Y.; Koteliansky, V.; Wolfe, S. A.; Langer, R.; Xue, W. & Anderson, D. G., “Therapeutic genome editing by combined viral and non-viral delivery of CRISPR system components in vivo”, Nature biotechnology 34, 328-333 (2016); Choi, J. G.; Dang, Y.; Abraham, S.; Ma, H.; Zhang, J.; Guo, H.; Cai, Y.; Mikkelsen, J. G.; Wu, H.; Shankar, P. & Manjunath, N., “Lentivirus pre-packed with Cas9 protein for safer gene editing”, Gene therapy 23, 627-633 (2016); Suresh, B.; Ramakrishna, S. & Kim, H., “Cell-Penetrating Peptide-Mediated Delivery of Cas9 Protein and Guide RNA for Genome Editing”, Methods in molecular biology (Clifton, N.J.) 1507, 81-94 (2017)]. Embodiments can co-deliver melt-guide, trans-activating nucleic acid and the NGEn endonuclease (as coding nucleic acid or protein) or separate their delivery. Components can also be stably expressed in cells. In some of these embodiments, expression of melt-guide can be achieved through reverse transcription.

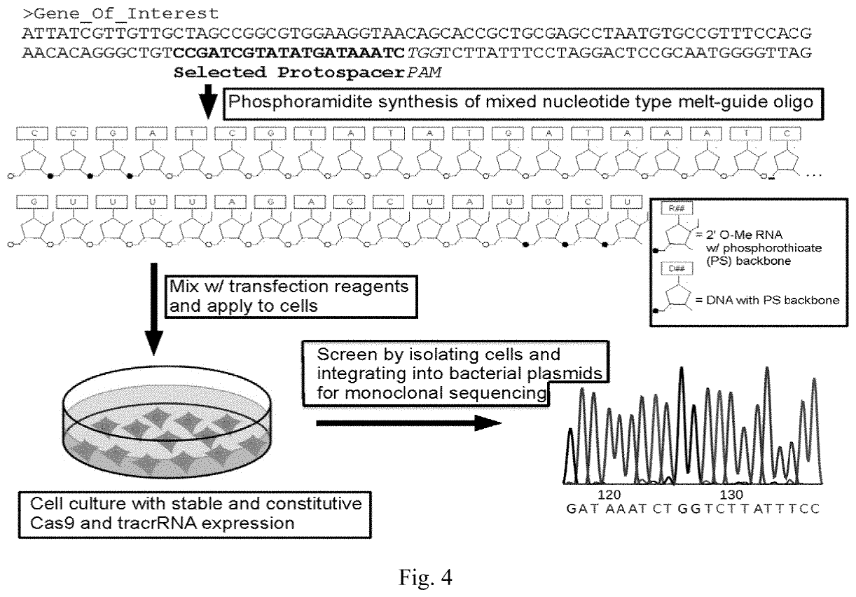

FIG. 4 depicts an example workflow to knockout a gene in cell culture stably expressing Cas9 and tracrRNA, according to one aspect of the invention. As illustrated in FIG. 4, the workflow starts with any preferred method for selecting a guide RNA target around a gene of interest from a fasta sequence file corresponding to this region. Selected protospacer sequences are used for designing the sequential phosphoramadite synthesis of a mixed nucleic acid type oligomer, keeping positions that would contact Cas9 at their 2′ hydroxl unmodified. The oligos are then mixed with transfection reagents (e.g. Lipofectamine RNAiMAX supplied by ThermoFisher) and applied to cell culture. After roughly two days of incubation, individual cells are isolated and regrown within separate wells on a tissue culture plate. Cells from each well are harvested for genomic extraction to allow an approximately one kilobase window around the target be amplified via polymerase chain reaction (PCR). The PCR product is ligated into a bacterial plasmid with a drug selection marker through blunt end cloning and transformed in E coli. Bacteria colonies are picked for monoclonal Sanger sequencing and can be carried out by services, such as Genewiz.

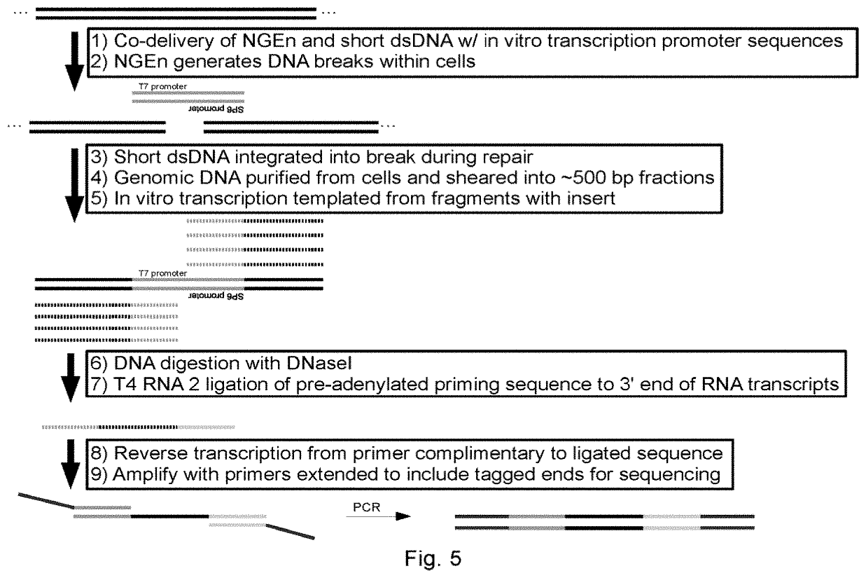

FIG. 5 depicts an example workflow for determining off-targets effects of guide molecules by introducing modifications to the Guide-seq protocol, according to one aspect of the invention. To determine off-targets effects of guide molecules, modifications are introduced to the Guide-seq protocol [Tsai, S. Q.; Zheng, Z.; Nguyen, N. T.; Liebers, M.; Topkar, V. V.; Thapar, V.; Wyvekens, N.; Khayter, C.; Iafrate, A. J.; Le, L. P.; Aryee, M. J. & Joung, J. K., “GUIDE-seq enables genome-wide profiling of off-target cleavage by CRISPR-Cas nucleases”, Nature biotechnology 33, 187-197 (2015)]. Short double stranded DNA is co-delivered with an NGEn system and integrates into genomic breaks. These short DNA duplexes can have overhangs with universal base-pairing nucleotides, such as deoxyinosine, for integration into overhang breaks. The inserted double stranded DNA contains inverted promoter sequences for in vitro transcription. Therefore when genomic DNA is purified and fractionated, the regions adjacent to insertion sites are transcribed by one of the promoters. A DNA oligo is ligated to the end of the transcripts or the transcripts are extended by polyadenylation, to enable their reverse transcription. Reverse transcription products are amplified by primers adding sequencing adapters.

A particular implementation of a protocol according to the invention for generating edited cells is as follows:

### Example Protocol for Melt-Guide Gene Targeting in Mammalian Cell Culture

1. For a target region of interest from a given genome, input the region's sequence into an online tool to design a 20 nt CRISPR Cas9 spacer.
2. Let represent the selected spacer sequence. From IDT order 100 nmol Alt-R™ CRISPR tracrRNA, 500 μg Alt-R™ S.p. Cas9 Nuclease 3NLS and a 100 nmol HPLC purified RNA oligo entered as:

N*N*N*NNNNNNNNNNNrNrNNNrNNmGrUrUrUmUmAmGmAmGmCmUm
AmUmGmCmU

3. Resuspend tracrRNA and melt-guide with nuclease-free water as 100 μM stock. Combine 1 μL of each with 98 μL IDT Duplex Buffer, heat the mixture to 90° C. and then gradually cool to room temperature to promote annealing between the two oligo species.
4. For each position in a 6-well cell culture plate, combine 125 μL ThermoFisher Opti-MEM™ Media, 2500 ng of Cas9 nuclease, 24 μL of the annealed oligos, 5 μL of ThermoFisher Lipofectamine™ Cas9 Plus™ Reagent.
5. For each well also mix 125 μL more media and 7.5 μL ThermoFisher Lipofectamine™ CRISPRMAX™ Reagent.
6. Wait 5 minutes before combining solutions from the previous two steps and then mix well.
7. Wait an additional 5-10 minutes before applying 250 μL of the mix from step 6 into a well with 30-70% confluent cell culture.
8. Incubate at 37° C. for 2-3 days before harvesting cells.

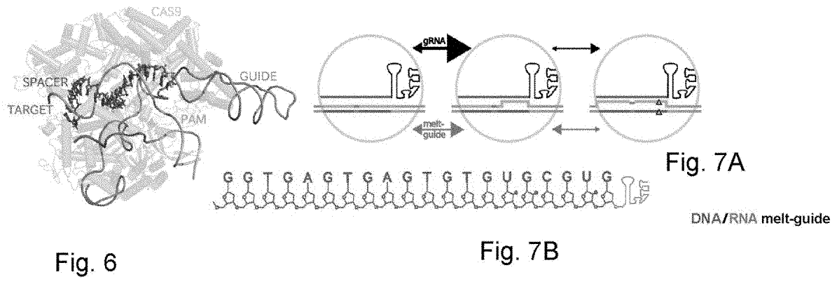

DNA substitutions in Cas9 gRNA improve mismatch sensitivity. Efforts that have measured and modeled Cas9 target recognition imply a mechanism that includes incremental strand invasion between gRNA spacer and target sequence [Farasat, I. & Salis, H. M., “A biophysical model of CRISPR/Cas9 activity for rational design of genome editing and gene regulation”, PLoS computational biology 12, e1004724 (2016); Josephs, E. A. et al., “Structure and specificity of the RNA-guided endonuclease cas9 during DNA interrogation, target binding and cleavage”, Nucleic Acids Research 43, 8924-8941 (2015)]. After prerequisite binding to a short protospacer adjacent motif (PAM), Cas9 helps stabilize DNA unwinding at a potential target as guide displaces its DNA:DNA base-pairs with RNA:DNA base-pairs (FIG. 6) [Jiang, F., Zhou, K., Ma, L., Gressel, S. & Doudna, J. A., “Structural biology. a Cas9-guide RNA complex preorganized for target DNA recognition”, Science 348, 1477-1481 (2015)]. After the resulting structure, called an R-loop, expands beyond a ^{˜}15 base-pair exchange, Cas9 can then create a double-strand DNA break [Jiang, F. et al., “Structures of a CRISPR-Cas9 r-loop complex primed for DNA cleavage”, Science 351, 867-871 (2016); Kiani, S. et al., “Cas9 gRNA engineering for genome editing, activation and repression”, Nature methods 12, 1051-1054 (2015)].

It is demonstrated that a Cas9 guide with DNA substitutions has reduced activity on mismatched targets. FIG. 6 depicts an example annotated 3D structure of a target-guide-Cas9 R-loop based on PDB 5F9R shown above the 2D structure of a melt-guide. In FIG. 6, red and yellow spheres highlight RNA 2′-hydroxyl groups retained and eliminated, respectively, in initial melt-guide designs.

Motivated by studies on RNA/DNA chimera hybridization indicating more DNA content generally decreased duplex stability, chimeric melt-guides promoting the rehybridization of mismatched R-loops were designed (FIGS. 7A-B) [Sugimoto, N. et al., “Thermodynamic parameters to predict stability of RNA/DNA hybrid duplexes”, Biochemistry 34, 11211-11216 (1995); Nakano, S.-i., Kanzaki, T. & Sugimoto, N., “Influences of ribonucleotide on a duplex conformation and its thermal stability: study with the chimeric RNA-DNA strands”, Journal of the American Chemical Society 126, 1088-1095 (2004)]. FIGS. 7A-B depicts a model of relative R-loop expansion rate differences (represented by arrow sizes and directions) that increase mismatch sensitivity for melt-guides compared to gRNA. Red segments indicate mismatches between guide and target. As illustrated in FIGS. 7A-B, candidate DNA-tolerant positions in gRNA were selected by excluding most positions containing RNA-specific 2′-hydroxyl contacts with Cas9 that may help maintain assembly of active OGN.

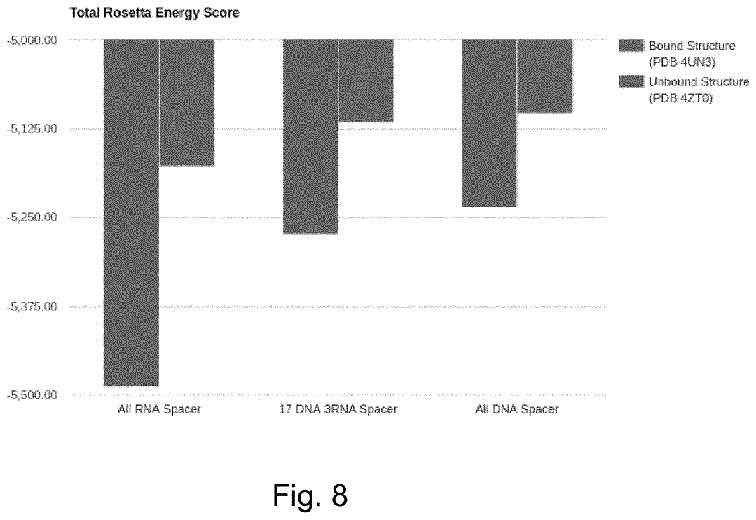

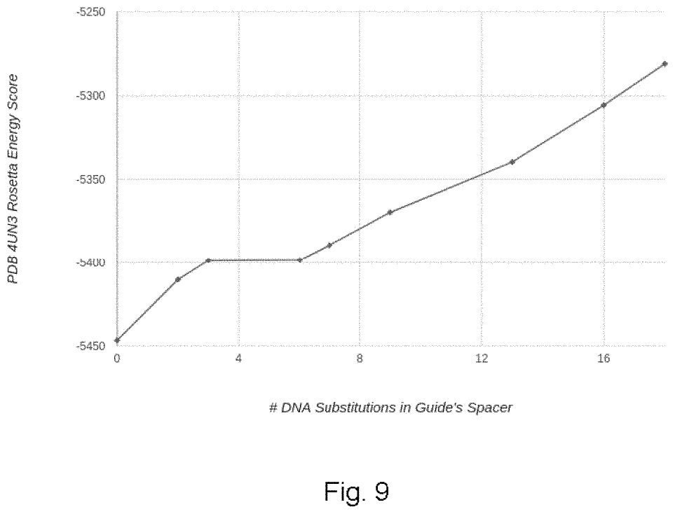

It was confirmed in silico via Rosetta that the selection strategy had a proportionally greater energy score penalty on published target-bound structures than for unbound guide-Cas9 [Nishimasu, H. et al., “Crystal structure of cas9 in complex with guide RNA and target DNA”, Cell 156, 935-949 (2014)]. FIGS. 8 and 9 depict Rosetta energy scores with DNA substitutions in bound and unbound structures from PDBs 4UN3 and 4ZTO. The interpretation that these scores, together, approximate R-loop stability and Cas9-guide affinity, led to substitution of most gRNA spacer bases with DNA.

For a standard target sequence from human VEGFA site 1, commercially synthesized chimeric melt-guides and corresponding on- and off-target DNA substrates were used to compare a melt-guide's mismatch discrimination to canonical gRNA when directing DNA cleavage. Table 1 lists sequence information with underlined mismatches.

TABLE 1

| Target Name | Sequence (Protospacer PAM) |
|---|---|
| VEGFA site 1 ON | GGTGAGTGAGTGTGTGCGTGTGG |
| | (SEQ ID No. 1) |
| VEGFA site 1 OFF1 | GGTGAGTGAGTGTGTGTGTGGGG |
| | (SEQ ID No. 2) |
| VEGFA site 1 OFF2 | GCTGAGTGAGTGTATGCGTGTGG |
| | (SEQ ID No. 3) |
| VEGFA site 1 OFF3 | TGTGGGTGAGTGTGTGCGTGAGG |
| | (SEQ ID No. 4) |
| VEGFA site 1 OFF4 | GGTGAACGAGTGTGTGCGTGTGG |
| | (SEQ ID No. 5) |
| VEGFA site 1 OFFS | GGTGAGTAGGTGTGTGCGTGTGG |
| | (SEQ ID No. 6) |
| VEGFA site 1 OFF6 | AGAGAGTGAGTGTGTGCATGAGG |
| | (SEQ ID No. 7) |

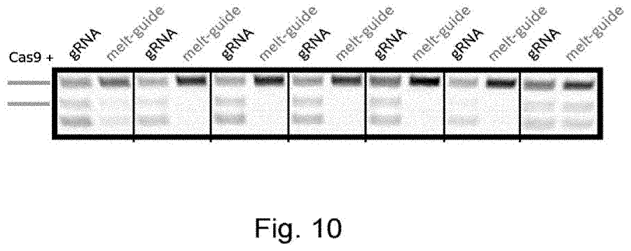

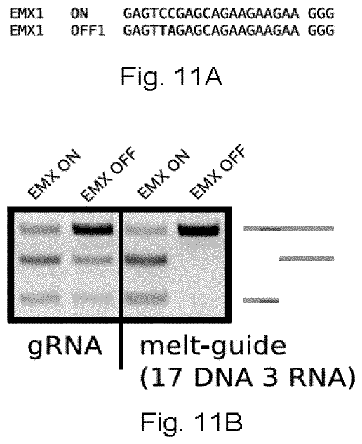

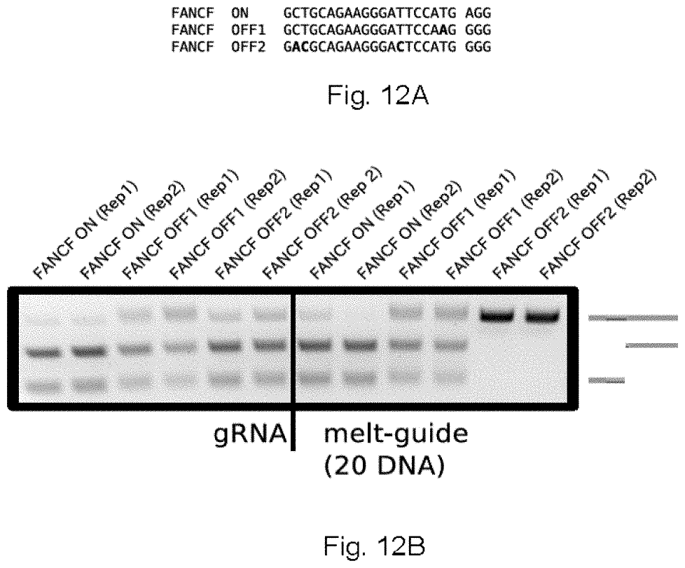

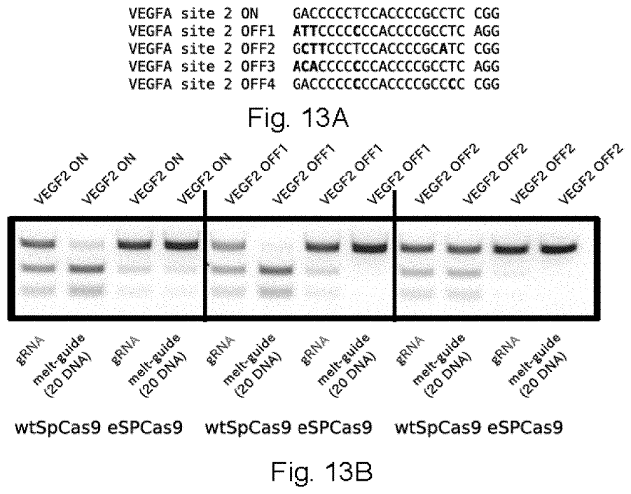

FIG. 10 is an inverted contrast-adjusted gel image of 4-hour Cas9 in vitro digests of targets with mismatches ranging from 0 to 3 using gRNA or melt-guide, shows that a melt-guide containing 17 DNA bases was functional in a 4-hour digestion assay with purified Cas9 and produced 74% the amount of cleaved on-target substrate as did gRNA. The same melt-guide resulted in no detectable cleavage for all surveyed two-mismatch off-targets, which, in many cases, gRNA-Cas9 cut faster than on-target substrate. Furthermore, on a challenging single-mismatch substrate that has been reported to be just as frequently an off-target for wild-type and high-fidelity enhanced SpCas9 (eSpCas9), the melt-guide reduced the digested fraction by four-fold [Kleinstiver, B. P. et al., “High-fidelity CRISPR-Cas9 nucleases with no detectable genome-wide off-target effects”, Nature 529, 490-495 (2016); Slaymaker, I. M. et al., “Rationally engineered Cas9 nucleases with improved specificity”, Science (New York, N.Y.) 351, 84-88 (2016)].

Additional in vitro assays demonstrate the generality of designing melt-guides for different genomic targets, but likewise reveal that targets comprising high GC and/or pyrimidine target content can limit sufficient destabilization to avoid cutting certain multi-mismatched sequences, even with melt-guides containing only DNA in the spacer [Gyi, J. I., Conn, G. L., Lane, A. N. & Brown, T., “Comparison of the thermodynamic stabilities and solution conformations of DNA.RNA hybrids containing purine-rich and pyrimidine-rich strands with DNA and RNA duplexes”, Biochemistry 35, 12538-12548 (1996)]. FIGS. 11A-B and 12A-B depict resulting gel images from Cas9 digests with melt-guides of on and off-target sequences for EA/X and FANCF, respectively. 13A-B depicts resulting gel images from Cas9 and eCas9 digests with melt-guides of on and off-target sequences for VEGFA site 2.

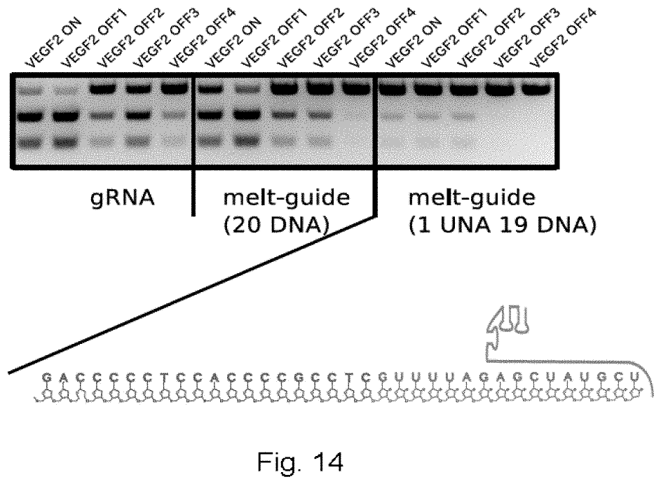

This limitation can be used to inform target-selection for a given application or it can be potentially overcome through combination of orthogonal destabilization techniques, such as truncating guide or complexing it with higher-fidelity Cas9 variants. FIG. 14 depicts gel images from Cas9 digests with melt-guides targeting VEGFA site 2 and Cas9 digests with additional UNA substitutions.

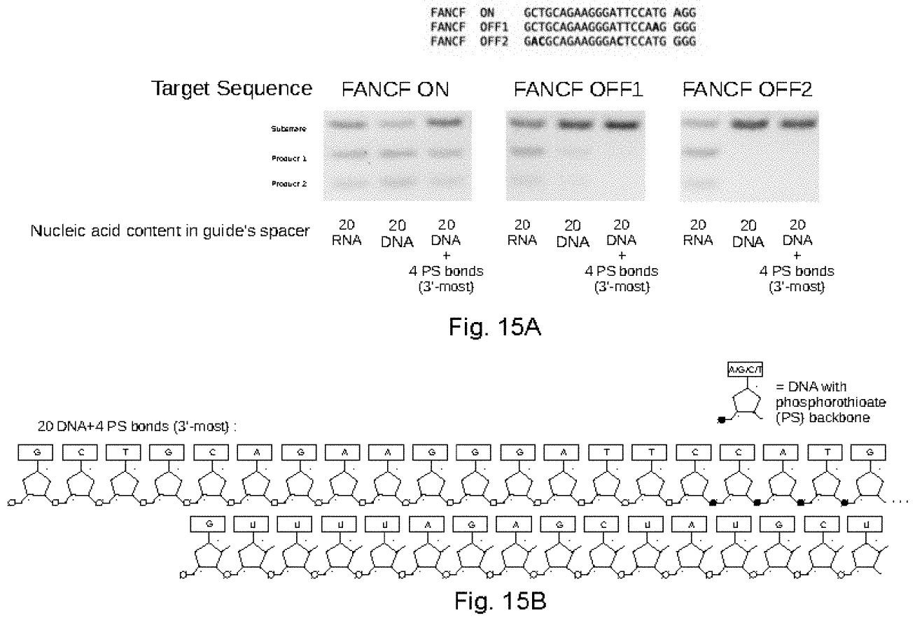

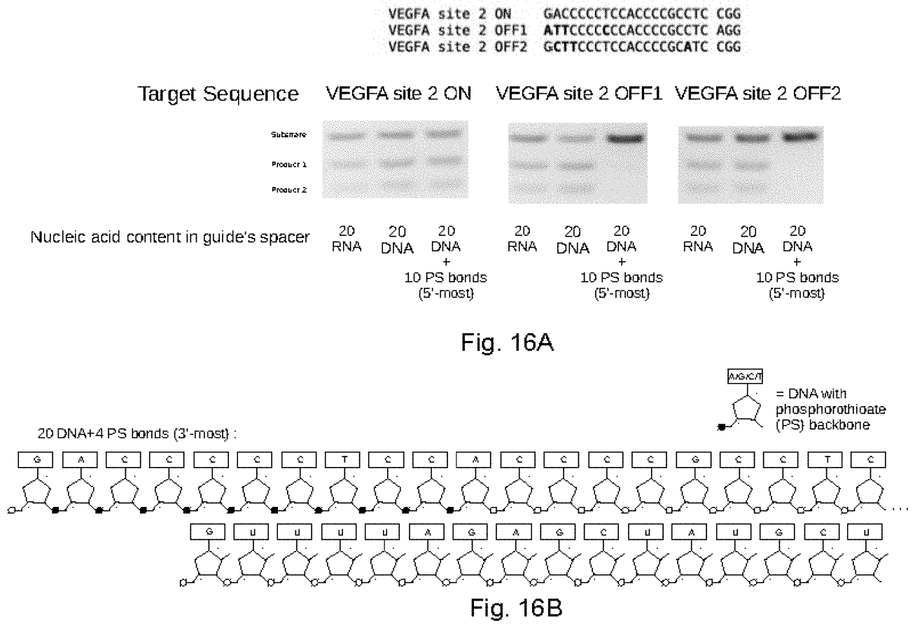

Other nucleotide-type substitutions that also enhance specificity have been identified, including unlocked nucleic acid (UNA) and abasic or universal base nucleotides at sequence positions with low-priority or no mismatches in the ensemble of possible off-targets [Snead, N. M., Escamilla-Powers, J. R., Rossi, J. J. & McCaffrey, A. P., “5′ unlocked nucleic acid modification improves sirna targeting”, Molecular therapy. Nucleic acids 2, e103 (2013); Zhang, J. et al., “Modification of the sirna passenger strand by 5-nitroindole dramatically reduces its off-target effects”, Chembiochem: a European journal of chemical biology 13, 1940-1945 (2012); Ghosh, M. K., Ghosh, K., Cohen, J. S., “Phosphorothioate-phosphodiester oligonucleotide co-polymers: assessment for antisense application”, Anticancer Drug Des., 8(1):15-32 (1993)]. FIGS. 15A-B and 16A-B demonstrate replacing phosphodiester bonds between bases with phosphorothioate bonds in positions of a guide's spacer that have potential to mismatch within a targeted genome can further enhance specificity, with FIGS. 15A-B depicting gel images from Cas9 digests with melt-guides targeting FANCF with 10 phosphorothioate (PS) bonds replacing phosodiester bonds and FIGS. 16A-B depicting gel images from Cas9 digests with melt-guides targeting VEGFA site 2 with 4 phosphorothioate (PS) bonds replacing phosodiester bonds.

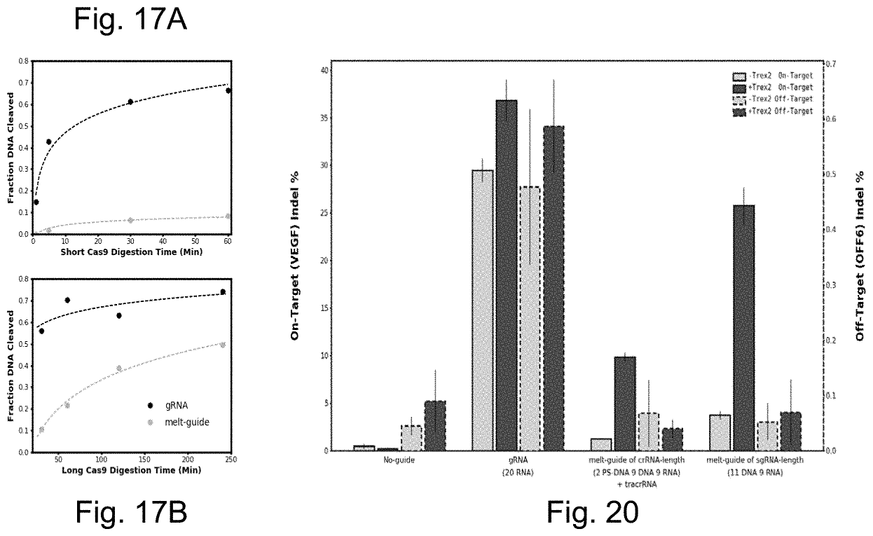

R-loop expansion kinetics determine melt-guide specificity. Whereas Cas9 is known to rapidly cleave DNA, its rates with melt-guides slowed appreciably [Sternberg, S. H., LaFrance, B., Kaplan, M. & Doudna, J. A., “Conformational control of DNA target cleavage by CRISPR-Cas9”, Nature 527, 110-113 (2015)]. Melt-guide strand invasion determines DNA cleavage and gene-editing rates. FIGS. 17A-B are graphs showing Melt-guide (gray) and gRNA (black) cleavage time courses of no-mismatch target with Cas9 plot alongside least-squares-fitting logarithmic functions (dashed curves).

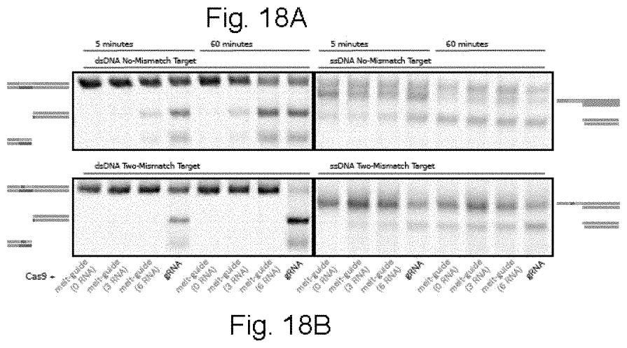

In order to confirm R-loop expansion contributes more than mismatched hybridization to this change in kinetics, time-coursed digestions were performed using substrates that were either double-stranded (ds) or single-stranded (ss) along the target. FIGS. 18A-B are inverted contrast-adjusted gel images of short (left-side within each quadrant) and long (right-side within each quadrant) Cas9 digests of double-stranded (left-half) and single-stranded (right-half) targets with no mismatches (FIG. 18A) or two (FIG. 18B) using gRNA or melt-guides with spacers containing either all-DNA, 3 DNA distributed, or an additional 3 DNA fill-in. Within minutes, Cas9 with canonical gRNA was able to cut both ds- and ss-target to near completion. For melt-guide-directed cleavage, steady digestion of no-mismatch ds-targets over several hours was observed, yet rates on ss-targets about as rapid as gRNA's and at similar timescales in the presence and absence of mismatches. The fast error-prone cuts detected upon removing strand-displacement from cleavage dynamics support that R-loop destabilization contributes mainly to melt-guides' improved specificity.

Future single-molecule fluorescent resonance energy transfer (FRET) measurements of melt-guides can be used to obtain finer detail of recognition kinetics and Cas9 conformational changes, complementary to previous work using gRNA [Singh, D., Sternberg, S. H., Fei, J., Doudna, J. A. & Ha, T., “Real-time observation of DNA recognition and rejection by the RNA-guided endonuclease Cas9”, Nature communications 7, 12778 (2016); Szczelkun, M. D. et al., “Direct observation of r-loop formation by single RNA-guided Cas9 and cascade effector complexes”, Proceedings of the National Academy of Sciences of the United States of America 111, 9798-9803 (2014)]. While it was noticed that melt-guides that include all-DNA-spacer did not introduce drastic structural changes that would have prevented cleavage, it is unclear whether such guides more closely adopt A-form or B-form duplexes with their target. This uncertainty arises from antagonistic influences of Cas9 pre-loading guide in an unpaired A—form versus the favored B-forming tendency of DNA:DNA dimers [Nishimasu, H. et al., “Crystal structure of Cas9 in complex with guide RNA and target DNA”, Cell 156, 935-949 (2014); Gyi, J. I., Lane, A. N., Conn, G. L. & Brown, T., “Solution structures of DNA.RNA hybrids with purine-rich and pyrimidine-rich strands: comparison with the homologous DNA and RNA duplexes”, Biochemistry 37, 73-80 (1998)]. The exact extent to which the helicity is altered for melt-guides in oligonucleotide-protein complexes could be solved from a crystal structure of the bound melt-guide OGN.

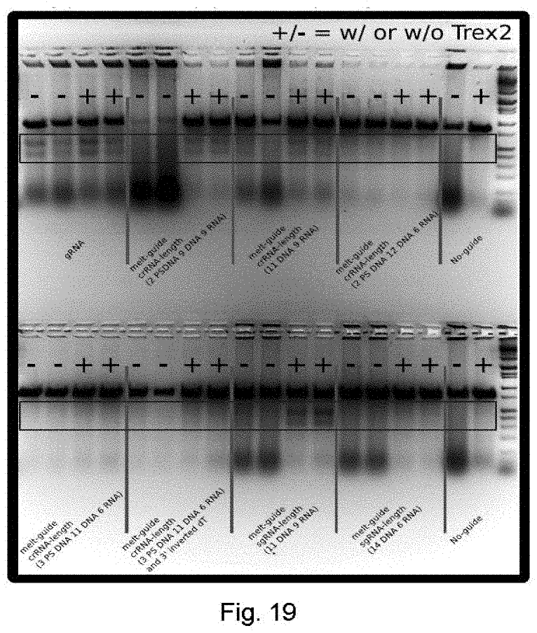

Melt-guide and Trex2 co-transfection reduces off-target genome editing. To test the use of melt-guides for genome editing, VEGFA site 1-targeting melt-guide oligos were transfected into HEK293T cells stably expressing Cas9 and enzymatically measured insertion/deletion (indel) mutations. FIG. 19 depicts the T7EI endonuclease assay on genomic VEGFA site 1 amplicons upon Cas9 mutagenesis with various melt-guide designs. Cleavage products in boxes are proportional to indel percentages. Initial attempts yielded unsatisfactorily low mutagenesis, which is believed to have resulted from unfavorable relative rates of: (i) guide oligo degradation, (ii) slower R-loop expansion, and (iii) errorless non-homologous end-joining (NHEJ) repair [Suzuki, K. et al., “In vivo genome editing via CRISPR/Cas9 mediated homology-independent targeted integration”, Nature 540, 144149 (2016); Schmid-Burgk, J. L., Hning, K., Ebert, T. S. & Hornung, V., “Crispaint allows modular base-specific gene tagging using a ligase-4-dependent mechanism”, Nature communications 7, 12338 (2016)]. Counteracting degradation with oligo lifetime-lengthening modifications (e.g., phosphorothioate (PS-DNA) or inverted terminal bases and 2′-O-methyl RNA substitutions on non-spacer guide positions) was tried, which partially restored cleavage rates by using fewer DNA substitutions in melt-guides [Hendel, A. et al., “Chemically modified guide RNAs enhance CRISPR-Cas genome editing in human primary cells”, Nature biotechnology 33, 985-989 (2015)]. Since these tactics did not lead to substantial improvement, methods were later pursued that could bias genomic double-strand breaks towards more error-prone repair.

Overexpression of the mammalian 3′ exonuclease Trex2, associated with DNA damage processing, has been reported to raise indel rates for various sequence-specific gene editing systems without causing toxicity [Delacte, F. et al., “High frequency targeted mutagenesis using engineered endonucleases and DNA-end processing enzymes”, PloS one 8, e53217 (2013); Certo, M. T. et al., “Coupling endonucleases with DNA end-processing enzymes to drive gene disruption”, Nature methods 9, 973-975 (2012); Chari, R., Mali, P., Moosburner, M. & Church, G. M., “Unraveling CRISPR-Cas9 genome engineering parameters via a library-on-library approach”, Nature methods 12, 823-826 (2015)]. Therefore, Trex2 expression plasmid was added to transfections and effected mutations were measured by deep sequencing [Pinello, L. et al., “Analyzing CRISPR genome-editing experiments with CRISPResso”, Nature biotechnology 34, 695-697 (2016)].

FIG. 20 is a dual y-axis chart showing deep sequencing indel measurements on-target and at a known off-target, comparing mutagenesis by gRNA to melt-guides designed with DNA substitutions in their first 11 positions with and without Trex2 overexpression (blue and light blue, respectively). Nucleic acid-type content in the guide's spacer is noted in parentheses. It was found that a melt-guide containing mostly DNA in spacer bases produced indel percentages above 25% on-target, which acceptably translates to 70% gRNA's rate. Crucially, on an off-target where gRNA-induced mutations were detected, melt-guides' indel percentages fell below the no-guide negative control. Between melt-guide types, single-molecule gRNA (sgRNA) length melt-guides consistently generated more than double the indel rate of melt-guides derived from shorter CRISPR RNA (crRNA) sequence, which need to duplex with trans-activating crRNA (tracrRNA). Despite Trex2 addition increasing indel percentages roughly seven-fold for both melt-guide types, the exonuclease had marginal impact on gRNA-directed mutation rates.

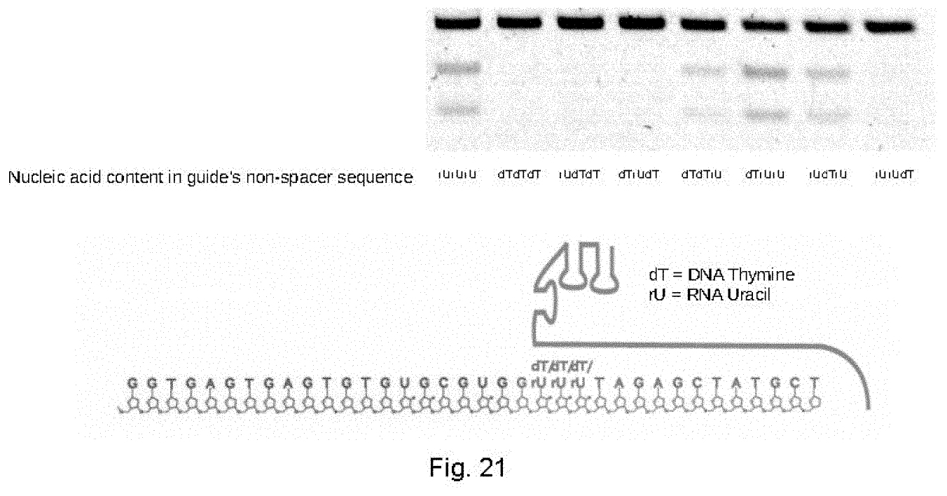

Others have achieved enhanced Cas9 specificity and could maintain high indel rates on-target without an accessory exonuclease [Kleinstiver, B. P. et al., “High-fidelity CRISPR-Cas9 nucleases with no detectable genome-wide off-target effects”, Nature 529, 490-495 (2016); Slaymaker, I. M. et al., “Rationally engineered Cas9 nucleases with improved specificity”, Science (New York, N.Y.) 351, 84-88 (2016)]. However, these experiments relied on transcribing all OGN components to abundant cellular concentrations. On one hand, a similar Trex2 supplementation strategy may benefit applications where some components are delivered as oligo or protein—which may include DNA-guided editing with Argonaute [Enghiad, B. & Zhao, H., “Programmable DNA-guided artificial restriction enzymes”, ACS synthetic biology 6, 752-757 (2017); Lee, S. H. et al., “Failure to detect DNA-guided genome editing using natronobacterium gregoryi argonaute”, Nature biotechnology 35, 17-18 (2016)]. On the other hand, a reverse-transcribable melt-guide with only DNA bases could lessen dependence on Trex2 for efficient mutagenesis. Towards that end, we show in vitro cleavage directed by tracrRNA in duplex with a crRNA-length melt-guide containing a single RNA outside of the spacer sequence. FIG. 21 is an inverted gel image of Cas9 digest of no-mismatch VEGFA site 1 target with almost all-DNA melt-guide of crRNA-length. Chimeras with such sparse RNA content are furthermore likely resistant to most Rnases.

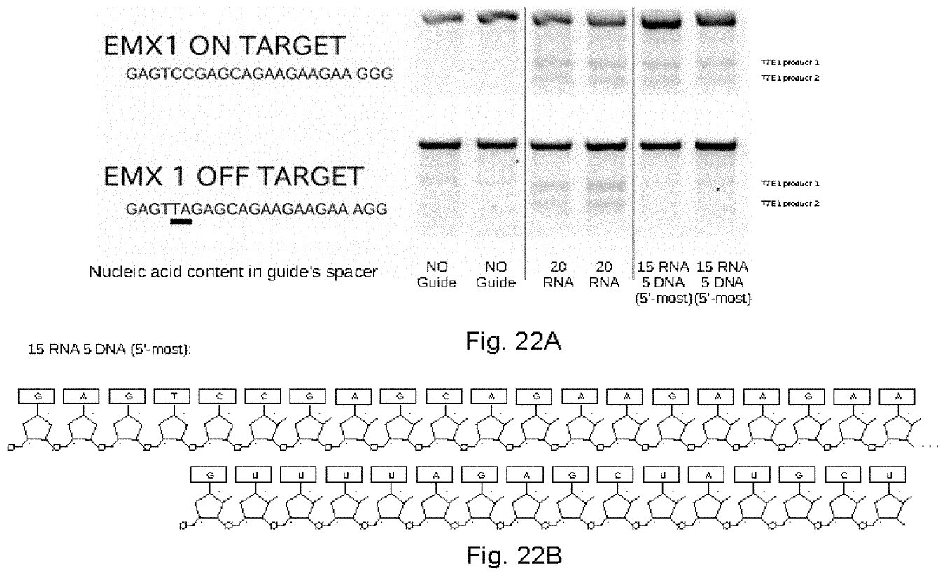

For certain targets, melt-guides achieve sufficient gene-editing efficiency in cells without the assistance of an exonuclease. FIGS. 22A-B depict a T7EI endonuclease assay on genomic EMX amplicons and a reported off-target upon mutagenesis by lipofection of protein Cas9 pre-assembled with either gRNA or melt-guide. The T7EI endonuclease assay of FIGS. 22A-B measures gene-editing on genomic EMX amplicons and those of its most-likely off-target upon Cas9 mutagenesis with either gRNA or Melt-guide containing 15 RNA bases and DNA bases. Similarly, by combining the specificity conferred by orthogonal specificity-enhancing methods, such as high-fidelity protein variants and truncated-guide RNA, the RNA/DNA content in a Melt-guide can be distributed to take advantage of when these methods permit synergistic, additive, or cooperative specificity.

Specific Experimental Methods.

Cas9-guide in vitro DNA digestions. Mixed nucleotide-type and RNA oligos, designed as Cas9 guides for selected standard genomic targets, were obtained from Integrated DNA Technologies (IDT). A 1 μM dilution was prepared for stocks of guide derived from sgRNA or crRNA and the latter was combined with equimolar tracrRNA (GE Dharmacon). Reactions consisted of 20 μM pre-annealed guide stock, 20 nM purified Cas9 from New England BioLabs (NEB) or purified eSpCas9 from Millipore Sigma, 10×NEB reaction buffer, and 500 ug of IDT-synthesized dsDNA target in 301 mixes. Samples were incubated at 37 Celsius and digested products separated by TAE-gel electrophoresis. Images of cleaved fractions from SYBR-Safe dsDNA gel stain (Thermo Fisher) under a blue light lamp were quantified using ImageJ software.

Preparation of single-stranded target DNA substrates. Target substrates were PCR-amplified using a primer oligo set (IDT) with 5′ phosphorylation for only the primer generating PAM-sided strands. Amplicons purified on anion-resin exchange columns (Qiagen) were digested by Lambda exonuclease (NEB), a 5′-to-3′ enzyme that prefers phosphorylated ends of dsDNA, to yield ssDNA of the strand opposite of PAM. Following subsequent column purification, ssDNAs were annealed to a primer beginning at the PAM site of the removed strand and templated for extension by DNA polymerase (NEB).

Genomic indel production and measurements. HEK293T cells stably expressing Cas9 purchased from GeneCopoeia were plated to 250,000 cells/35 mm well in 2.5 ml Dulbeccos Modified Eagles Medium with 10% Fetal Bovine Serum and incubated at 37 Celsius and 5% CO2. The next day, transfections via TransIT-X2 reagent (Minis Bio) delivered a 25 nM final concentration of guide with or without 2.5 pExodus CMV.Trex2, which was a gift from Dr. Andrew Scharenberg (Addgene plasmid #40210). After an additional 48 hours, genomic DNA was isolated using Epicentre QuickExtract solution and indel production was visualized by a common T7 Endonuclease I assay (NEB) on amplicons from on-target and known off-target regions that were denatured and re-annealed [Vouillot, L., Thlie, A. & Pollet, N., “Comparison of T7E1 and surveyor mismatch cleavage assays to detect mutations triggered by engineered nucleases”, G3 5, 407-415 (2015)]. Amplicons were then prepared for deep sequencing with Nextera-XT tagmentation (Illumina) and run on a MiSeq 2×300 v3 kit (Illumina). Reads were analyzed using the CRISPResso software pipeline for precise indel percentages from biological and technical duplicates [Pinello, L. et al., “Analyzing CRISPR genome-editing experiments with CRISPResso”, Nature biotechnology 34, 695-697 (2016)].

In cases when protein Cas9 was delivered, HEK293T cells were cultured in 6-well plates at a density of 5×10⁵/well in Advanced DMEM media (ThermoFisher) supplemented with 10% FBS, 2 mM GlutaMax (ThermoFisher), and penicillin/streptomycin, at 37° C. with 5% CO2. After 24 hours, either melt-guides or control guides (100 nM) and Cas9 RNP (2.5 μg) were first complexed with Cas9 Plus reagent (ThermoFisher) in Opti-MEM (Gibco) for 10 minutes and subsequently mixed with the CRISPRMAX lipofectamine (ThermoFisher) reagent for 10 minutes, and transfected into cells. After 48 hours of transfection, cells were harvested and genomic DNA was isolated using 100 μl of QuickExtract (EpiCentre) solution. On-target or off-target loci were amplified for indel analysis using the T7E1 mismatch-sensitive endonuclease.

In the case of Cas9, the precision of target activity in vitro and in vivo was improved with mismatch-evading lowered-thermostability guides. Melt-guides should be extensible to the expanding collection of CRISPR systems by extrapolating either from chimeric oligo libraries to scan nucleotide-type substitution or from published crystal structure data to avoid disrupting RNA-specific interactions (i.e., Cpf1 guide's pseudoknots) [Yamano, T. et al., “Crystal structure of cpfl in complex with guide RNA and target DNA”, Cell 165, 949-962 (2016); Burstein, D. et al., “New CRISPR-Cas systems from uncultivated microbes”, Nature 542, 237-241 (2017)]. Given the minimal RNA content that was found to be sufficient for guiding Cas9, additional protein engineering, perhaps through homolog alignments, may enable the realization of all-DNA melt-guides.

Just as keeping some of the nucleotide type content of a Melt-guide's spacer as RNA can be employed to support distributions of thermostability-lowering nucleotide types and base linkages in the spacer that maintain catalytic efficiency and improved specificity, additional substitutions consisting of nucleotide types that instead increase thermostability, such as locked nucleic acid (LNA), could also support destabilizing substitutions distributed elsewhere within the Melt-guide to restore or improve catalytic effectiveness [Owczarzy, Richard, You, Yong, Groth, Christopher L., and Tataurov, Andrey V., “Stability and Mismatch Discrimination of Locked Nucleic Acid-DNA Duplexes”, Biochemistry 50(43): 9352-9367 (2011)]

The invention demonstrates that a DNA:RNA chimeric guide strand is sufficient to guide Cas9 to a specified target sequence for indel formation and minimize off-target cleavage events due to the specificity conferred by DNA-DNA interactions. The invention provides a novel strategy of precision genome engineering utilizing the CRISPR-Cas9 gene editing system. It also has the potential for a positive translational impact by minimizing the risks associated with human genomic modifications in clinical settings. Overall, these findings are expected to catalyze an expanding synthetic genome engineering research program, involving both mechanistic follow-up studies and novel approaches to gene editing using alternate guide molecules and engineered endonucleases.

While preferred embodiments of the invention are disclosed herein, many other implementations will occur to one of ordinary skill in the art and are all within the scope of the invention. Each of the various embodiments described above may be combined with other described embodiments in order to provide multiple features. Furthermore, while the foregoing describes a number of separate embodiments of the apparatus and method of the present invention, what has been described herein is merely illustrative of the application of the principles of the present invention. Other arrangements, methods, modifications, and substitutions by one of ordinary skill in the art are therefore also considered to be within the scope of the present invention.

What is claimed is:

1. A method for genome editing, comprising:
generating a complex comprising Cas9, trans-activating CRISPR RNA, and a chimeric CRISPR RNA having a target-defining spacer region comprising a plurality of DNA nucleotides and a plurality of RNA bases, wherein the guide promotes sufficient strand invasion by the RNA motif to allow the complex to be active while also exhibiting increased cleavage specificity via the less thermodynamically stable DNA-DNA interaction; and
co-transfecting the complex into a cell culture with an exonuclease that promotes DNA repair.

2. The method of claim 1, wherein a majority of the bases in the modified CRISPR RNA guide's target-defining spacer region are DNA nucleotides.

3. An edited genome produced by the method of claim 2.

4. A method for generating a chimeric DNA:RNA guide strand for genome editing, comprising replacing a plurality of bases in the target-defining spacer region of a CRISPR RNA guide with DNA nucleotides.

5. The method of claim 4, wherein the majority of the bases in the CRISPR RNA guide's target-defining spacer region have been swapped with DNA nucleotides.

6. The method of claim 4, wherein the spacer sequence of the guide primarily consists of DNA, while the spacer bases that interact with Cas9 and trans-activating CRISPR RNA remain as RNA.

7. A chimeric DNA:RNA guide strand produced by the method of claim 5.

8. A method for reducing off-target CRISPR-Cas9 cleavage events in genome editing, comprising:
generating a chimeric DNA:RNA guide strand for genome editing, comprising replacing a plurality of bases in the target-defining spacer region of a CRISPR RNA guide with DNA nucleotides;
generating a complex comprising Cas9, trans-activating CRISPR RNA, and a chimeric CRISPR RNA having a target-defining spacer region comprising a plurality of DNA nucleotides and a plurality of RNA bases, wherein the guide promotes sufficient strand invasion by the RNA motif to allow the complex to be active while also exhibiting increased cleavage specificity via the less thermodynamically stable DNA-DNA interaction; and
co-transfecting the complex into a cell culture with an exonuclease that promotes DNA repair.

9. The method of claim 8, wherein a majority of the bases in the CRISPR RNA guide's target-defining spacer region are DNA nucleotides.

10. The method of claim 8, wherein a majority of those bases in the target-defining spacer region of the guide that interact with the target are DNA nucleotides, while the bases that interact with Cas9 and trans-activating CRISPR RNA are RNA nucleotides.


SEQUENCE LISTING (30 sequences)

SEQ ID NO: 1 - DNA, 23 bp, Unknown
  Protospacer PAM
ggtgagtgagtgtgtgcgtgtgg

SEQ ID NO: 2 - DNA, 23 bp, Unknown
  Protospacer PAM
ggtgagtgagtgtgtgtgtgggg

SEQ ID NO: 3 - DNA, 23 bp, Unknown
  Protospacer PAM
gctgagtgagtgtatgcgtgtgg

SEQ ID NO: 4 - DNA, 23 bp, Unknown
  Protospacer PAM
tgtgggtgagtgtgtgcgtgagg

SEQ ID NO: 5 - DNA, 23 bp, Unknown
  Protospacer PAM
ggtgaacgagtgtgtgcgtgtgg

SEQ ID NO: 6 - DNA, 23 bp, Unknown
  Protospacer PAM
ggtgagtaggtgtgtgcgtgtgg

SEQ ID NO: 7 - DNA, 23 bp, Unknown
  Protospacer PAM
agagagtgagtgtgtgcatgagg

SEQ ID NO: 8 - RNA, 16 nt, Unknown
  crRNA 3′ universal handle
guuuuagagcuaugcu

SEQ ID NO: 9 - DNA, 20 bp, Unknown
  meltGuide
ggtgagtgagtgtgugcgug

SEQ ID NO: 10 - RNA, 67 nt, Unknown
  tracrRNA
agcauagcaaguuaaaauaaggcuaguccguuaucaacuugaaaaaguggcaccgagucggugcuuu

SEQ ID NO: 11 - DNA, 20 bp, Unknown
  meltGuide
ggtgagtgagtgtgtgcgug

SEQ ID NO: 12 - DNA, 20 bp, Unknown
  meltGuide
ggtgagtgagtgtgtgcgtg

SEQ ID NO: 13 - DNA, 20 bp, Unknown
  meltGuide
ggtgagtgagtgtgugcgtg

SEQ ID NO: 14 - DNA, 136 bp, Unknown
  target gene
attatcgttgttgctagccggcgtggaaggtaacagcaccgctgcgagcctaatgtgccgtttccacgaacacagggctgtccgatcgtatatgataaatctggtcttatttcctaggactccgcaatggggttag

SEQ ID NO: 15 - DNA, 36 bp, Unknown
  meltGuide
ccgatcgtatatgataaatcguuuuagagcuaugcu

SEQ ID NO: 16 - DNA, 21 bp, Unknown
  modified target gene
gataaatctggtcttatttcc

SEQ ID NO: 17 - DNA, 23 bp, Unknown
  Protospacer PAM
gagtccgagcagaagaagaaggg

SEQ ID NO: 18 - DNA, 23 bp, Unknown
  Protospacer PAM
gagttagagcagaagaagaaggg

SEQ ID NO: 19 - DNA, 23 bp, Unknown
  Protospacer PAM
gctgcagaagggattccatgagg

SEQ ID NO: 20 - DNA, 23 bp, Unknown
  Protospacer PAM
gctgcagaagggattccaagggg

SEQ ID NO: 21 - DNA, 23 bp, Unknown
  Protospacer PAM
gacgcagaagggactccatgggg

SEQ ID NO: 22 - DNA, 23 bp, Unknown
  Protospacer PAM
gaccccctccaccccgcctccgg

SEQ ID NO: 23 - DNA, 23 bp, Unknown
  Protospacer PAM
attcccccccaccccgcctcagg

SEQ ID NO: 24 - DNA, 23 bp, Unknown
  Protospacer PAM
gcttccctccaccccgcatccgg

SEQ ID NO: 25 - DNA, 23 bp, Unknown
  Protospacer PAM
acacccccccaccccgcctcagg

SEQ ID NO: 26 - DNA, 23 bp, Unknown
  Protospacer PAM
gaccccccccaccccgcccccgg

SEQ ID NO: 27 - DNA, 36 bp, Unknown
  meltGuide
gaccccctccaccccgcctcguuuuagagcuaugcu

SEQ ID NO: 28 - DNA, 36 bp, Unknown
  meltGuide
gctgcagaagggattccatgguuuuagagcuaugcu

SEQ ID NO: 29 - DNA, 36 bp, Unknown
  meltGuide
ggtgagtgagtgtgugcgugguuutagagctatgct

SEQ ID NO: 30 - DNA, 36 bp, Unknown
  meltGuide
gagtccgagcagaagaagaaguuuuagagcuaugcu